(19) United States
(12) Patent Application Publication
(10) Pub. No.: US 2023/0149176 A1
(43) Pub. Date: May 18, 2023

(54) Multi-Direction Fixation for Shoulder Prosthesis

(71) Applicant: Howmedica Osteonics Corp., Mahwah, NJ (US)

(72) Inventors: Sunny Shorabh, Ghaziabad (IN); Vishal Dilip Jagtap, Thane (IN); Andrew J. Nelson, New City, NY (US); Rajan Yadav, New Delhi (IN)

(21) Appl. No.: 17/986,133

(22) Filed: Nov. 14, 2022

Related U.S. Application Data
(60) Provisional application No. 63/279,711, filed on Nov. 16, 2021.

(51) Int. Cl. A61F 2/40

(52) U.S. Cl. CPC A61F 2/4014; A61F 2002/4022; A61F 2002/30331

(57) ABSTRACT

According to one aspect of the disclosure, a prosthetic implant system includes a first articulation component, a base, and a second anchor. The base may have a proximal portion and a first anchor extending in a distal direction along a longitudinal first anchor axis. The proximal portion of the base may be configured to couple to the first articulation component. The second anchor may be formed separately from the base and may extend along a longitudinal second anchor axis. The base may include a channel extending from a first opening in the proximal portion of the base through a second opening in a distal portion of the first anchor. The channel may be sized and shaped to receive the second anchor therethrough. When the second anchor is received within the channel, the longitudinal first anchor axis may be oblique to the longitudinal second anchor axis.

### CROSS-REFERENCE TO RELATED APPLICATIONS

The present application claims the benefit of the filing date of U.S. Provisional Application No. 63/279,711, filed on Nov. 16, 2021, the disclosure of which is hereby incorporated herein by reference.

### FIELD OF THE DISCLOSURE

The present application generally relates to a shoulder prosthesis and a method of implantation of a shoulder prosthesis, although the concepts described herein are applicable to other joint prostheses.

### BACKGROUND OF THE DISCLOSURE

Over time and through repeated use, bones and joints can become damaged or worn. For example, repetitive strain on bones and joints (e.g., through athletic activity), traumatic events, and certain diseases (e.g., arthritis) can cause cartilage in joint areas, for example, which normally provides a cushioning effect, to wear down. When the cartilage wears down, fluid can accumulate in the joint areas, resulting in pain, stiffness, and decreased mobility. The same can happen in cases where tendons in a joint become lax or soft tissues in or adjacent the joint become damaged or worn.

Arthroplasty procedures can be used to repair such damaged joints. During a typical arthroplasty procedure, an arthritic or otherwise dysfunctional joint can be remodeled or realigned. A prosthesis or prostheses can be implanted to repair the damaged region(s). Arthroplasty procedures may take place in any of a number of different regions of the body, such as the knees, hips, shoulders, or elbows, for example. One type of arthroplasty procedure is a shoulder arthroplasty, in which a damaged shoulder joint may be replaced with prosthetic implants. The shoulder joint may have been damaged by, for example, arthritis (e.g., severe osteoarthritis or degenerative arthritis), trauma, or a destructive joint disease.

Shoulder prostheses may take the form of anatomic or reverse implants. In anatomic shoulder implants, the native humeral head is replaced with a prosthetic humeral head, and/or the native glenoid is replaced with a prosthetic glenoid. In a reverse shoulder implant, the native humeral head is replaced with a prosthetic cup or socket component, and the native glenoid is replaced with a prosthetic ball component (e.g. a glenosphere). As the name suggests, a reverse prosthetic shoulder system reverses the positions of the ball and socket components of the joint relative to the native positions of those components.

Prostheses that are implanted into a damaged region may provide support and structure to the damaged region, and may help to restore the damaged region, thereby enhancing its functionality. Prior to implantation of a prosthesis in a damaged region, the damaged region may be prepared to receive the prosthesis. In the case of a shoulder arthroplasty procedure, one or more of the bones in the shoulder area, such as the humerus and/or glenoid, may be treated (e.g., cut, drilled, reamed, and/or resurfaced) to provide one or more surfaces that can align with the implant and thereby accommodate the implant.

The fixation of an implant component into the bone is typically an important features of the implant system. Joint implants are frequently under loads of varying amounts and in varying positions. If an implant component becomes loose, it can become ineffective and even need to be removed and replaced with a new implant. Thus, fixation is an important feature of joint implant systems. For stemless humeral implants, fixation can be particularly important because stemless humeral implants typically have less structure than corresponding stemmed humeral implants, which may make proper fixation more difficult to achieve. However, proper fixation is important for all joint implants, including both stemmed and stemless humeral implants.

### BRIEF SUMMARY OF THE DISCLOSURE

According to one aspect of the disclosure, a prosthetic implant system includes a first articulation component, a base, and a second anchor. The base may have a proximal portion and a first anchor extending in a distal direction along a longitudinal first anchor axis. The proximal portion of the base may be configured to couple to the first articulation component. The second anchor may be formed separately from the base and may extend along a longitudinal second anchor axis. The base may include a channel extending from a first opening in the proximal portion of the base through a second opening in a distal portion of the first anchor. The channel may be sized and shaped to receive the second anchor therethrough. When the second anchor is received within the channel, the longitudinal first anchor axis may be oblique to the longitudinal second anchor axis.

### BRIEF DESCRIPTION OF THE DRAWINGS

FIG. 1 is a view of bones of a shoulder joint.

FIG. 2 is a view of the bones of the shoulder joint of FIG. 1 along with representations of the subscapularis muscle and the supraspinatus muscle.

FIGS. 3A-C are cross-sections of different steps of implanting a base of a prosthetic humeral implant into a humerus.

FIG. 3D is a cross-section of a base and secondary anchor implanted into a humerus, the secondary anchor being elongated relative to that shown in FIGS. 3A-C.

FIG. 4A is a perspective view of the base of the prosthetic humeral implant, and secondary anchor, shown in FIGS. 3A-C but provided with additional detail.

FIG. 4B is a side view of the base illustrated in FIG. 4A.

FIG. 4C is a cross-section of base of FIG. 4B taken along the section line 4C-4C of FIG. 4B.

FIG. 5A is a perspective view of components of a humeral implant system according to another embodiment of the disclosure.

FIG. 5B is a cross-section of the base and secondary anchor of FIG. 5A implanted into a humerus.

FIG. 6A is a perspective view of components of a humeral implant system according to a further embodiment of the disclosure.

FIG. 6B is a side view of the humeral implant system of FIG. 6A assembled and implanted into a humerus.

### DETAILED DESCRIPTION

As used herein, the term “proximal” refers to a location closer to an individual's heart, and the term “distal” refers to a location farther away from the individual's heart. When used in the context of an implant, the terms “proximal” and “distal” refer to locations on the implant closer to, or farther away from, the heart when the implant is implanted in an intended manner. As used herein, the term “medial” refers to a location closer to the midline of an individual, while the term “lateral” refers to allocation farther away from the midline of the individual. Further, it should be understood that although the term “stemless implant” is used herein, the term does not indicate that a stemless implant fully lacks any anchor, but rather a stemless implant may include an anchor that is significantly smaller and/or shorter than stems of typical known stemmed implants.

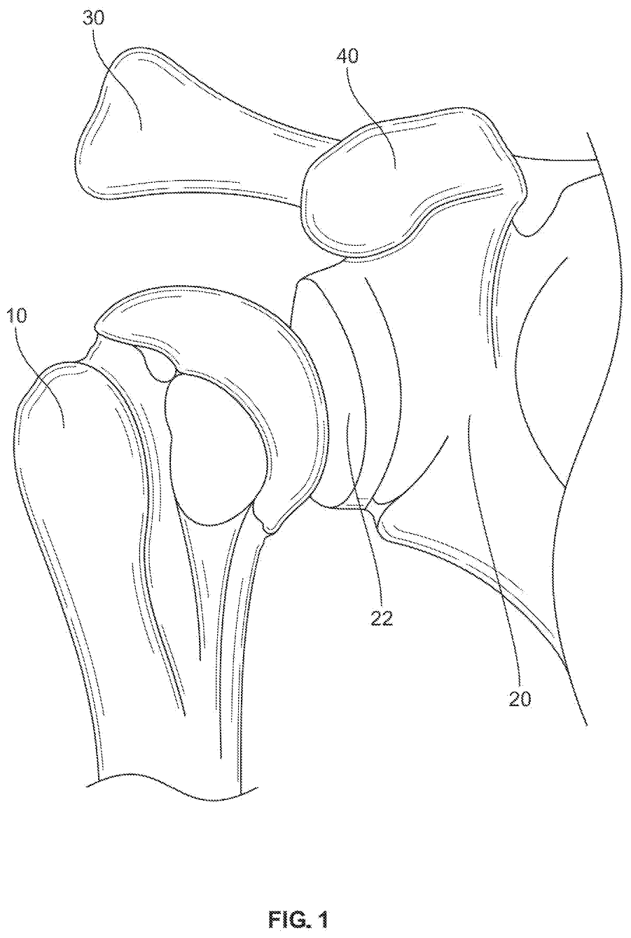

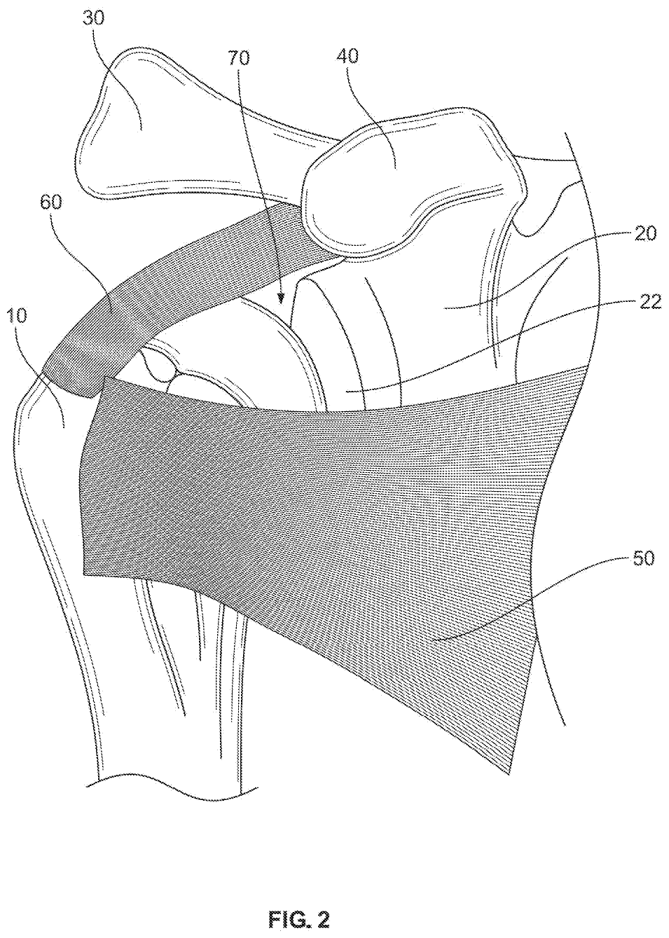

FIG. 1 illustrates bones of a right shoulder joint as viewed anteriorly (from the front of a patient). Generally, the bones of the shoulder joint include humerus 10, a proximal end of which includes a head or ball of the ball-and-socket joint, and scapula 20, which includes the glenoid cavity 22 that forms the socket of the ball-and-socket joint. The acromion 30 and coracoid process 40 are also illustrated in FIG. 1. FIG. 2 illustrates the bones of FIG. 1 but also provides representations of the subscapularis muscle 50 and the supraspinatus muscle 60. Generally, the subscapularis muscle 50 is attached to the lesser tubercle of the humerus 10, and the supraspinatus muscle 60 is attached to the greater tubercle of the humerus 10. The subscapularis muscle 50 and supraspinatus muscle 60 form two of the four muscles forming the rotator cuff. As shown in FIG. 2, a generally triangular space is formed between (i) the tendons of the subscapularis muscle 50, (ii) the tendons of the supraspinatus muscle 60, and (iii) the base of the coracoid process 40. This triangular space is known as the rotator cuff interval 70. It should be understood that FIG. 2 is not intended to be a complete representation of all soft tissue pertinent in a shoulder joint.

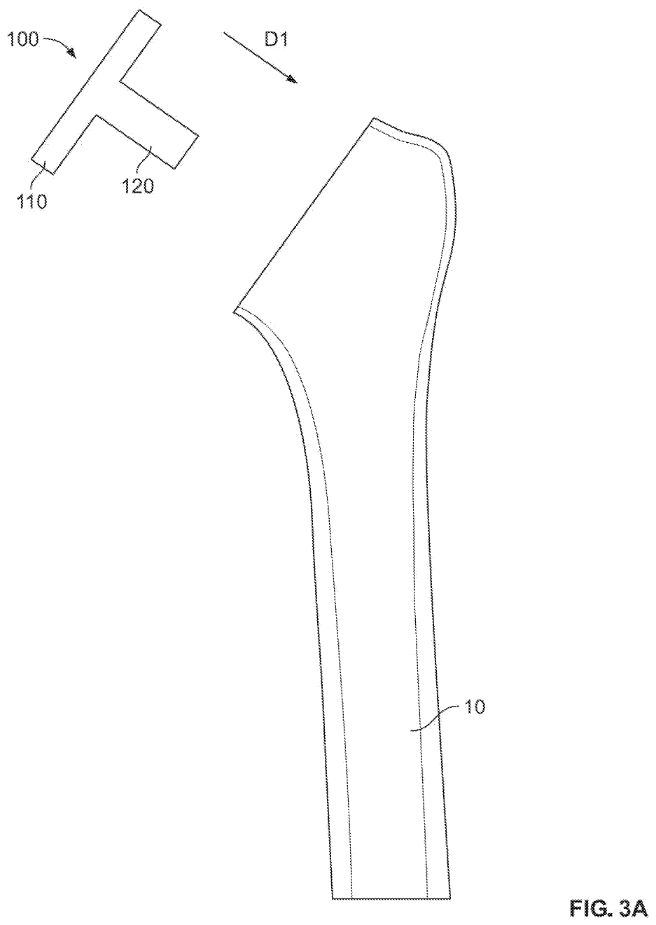

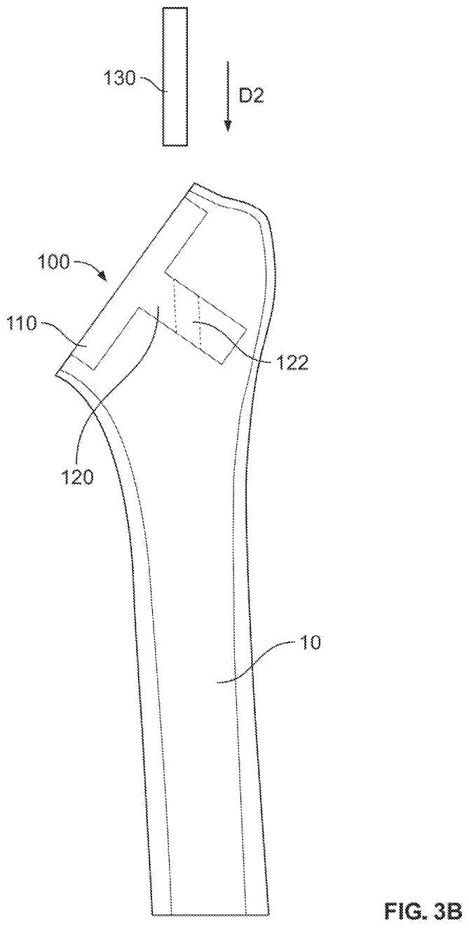

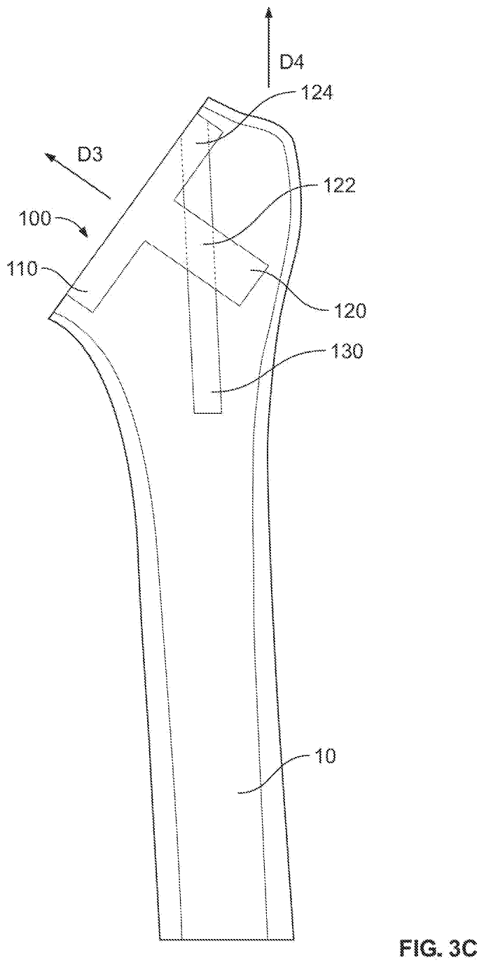

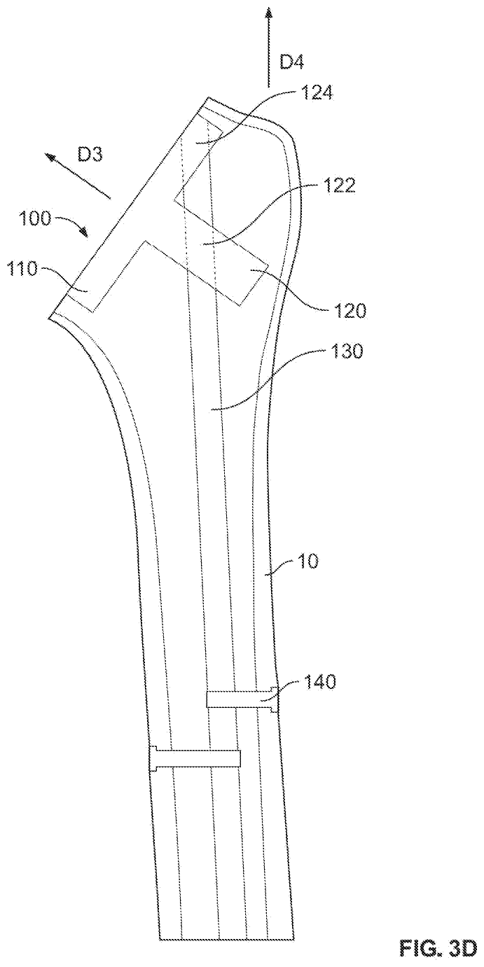

One aspect of a shoulder arthroplasty may include removal of the head of the humerus 10 and replacement of the humeral head with a prosthetic humeral head (in an anatomic shoulder implant) or a prosthetic cup (in a reverse shoulder implant). For example, FIGS. 3A-C illustrate different steps of implanting a base 100 of a prosthetic humeral implant. It should be understood that the base 100 may be configured to receive a prosthetic humeral head for an anatomic shoulder replacement, a prosthetic cup for a reverse shoulder replacement, or otherwise any suitable component of an anatomic or reverse shoulder replacement.

In FIG. 3A, the native humeral head has already been cut to provide a generally planar surface in which base 100 will be received. Generally, base 100 may include a collar 110 and an anchor 120. The collar 110 may be generally circular, although it may take other shapes, such as a triangular shape, oblong shape, or a shield-shape. The proximal surface of the collar 110 is preferably planar, and when implanted, the proximal planar surface of the collar 110 may substantially align with the proximal planar resected surface of the humerus, although in other embodiments the surface of the collar need not directly align with the surface of the proximal humeral resection. The anchor 120 extends distally from the collar 110 and generally serves to provide an amount of fixation between the base 100 and the humerus 10. In the illustrated embodiment, the anchor 120 is illustrated in a simplified format, but the anchor 120 may take various shapes. In some embodiments, the anchor 120 extends distally from the collar 110 along a central longitudinal axis that is substantially perpendicular to the proximal surface of the collar 110. After making the proximal humeral resection, the base 100 may be implanted into the humerus 10 by any suit able method. In some embodiments, the proximal humerus may be further prepared, e.g. via drilling pilot holes or via punch mechanisms to provide complementary or guiding shapes to receive the base 100. In other embodiments, the base 100 may be implanted without further preparation, for example by driving the base 100 into the proximal humerus the desired distance, for example until the proximal surface of the collar 110 is about flush with the proximal humeral resection. In the embodiment shown in FIG. 3A, the base 100 is driven in direction D1, which may be generally parallel the longitudinal axis of the anchor 120 and/or perpendicular the proximal resected surface of the humerus 10 (and/or perpendicular the proximal surface of the collar 110), until the base 100 is positioned at the desired depth and angle, as shown in FIG. 3B.

Now referring to FIG. 3B, after the base 100 has been implanted, a secondary anchor 130 may be implanted into the humerus 10 and engaged with the base 100, for example through the anchor 120. In the particular embodiment illustrated in FIG. 3B, anchor 120 includes a channel 122 sized to accept the secondary anchor 130 therethrough. For example, the channel 122 may extend in a direction so that, when the base 100 is implanted, the channel 122 is substantially parallel with (or even generally coaxial with) a longitudinal axis of the intramedullary canal of the humerus 10. However, it should be understood that other relative angles may also be suitable. Preferably, the channel 122 is formed so that structure of the anchor 120 circumscribes the channel 122 fully, although in other embodiments a less-than-complete circumscription may be suitable. The cross-sectional shape of the channel 122 preferably matches the cross-sectional shape of the secondary anchor 130. For example, if the secondary anchor 130 is cylindrical (with a circular cross-section), the channel 122 is preferably cylindrical (with a circular cross-section). The cross-sections of the channel 122 and the secondary anchor 130 may be other than circular, including oblong, triangular, rectangular, square, etc. Preferably, the dimensions of the channel 122 match or are slightly larger than those of the secondary anchor 130 so that the secondary anchor 130 has a tight fit with the channel. In some embodiments, locking features may be provided, such as matching threading between the channel 122 and the secondary anchor 130 so that the secondary anchor 130 may be screwed into and through the channel 122. As shown in FIG. 3B, the secondary anchor 130 may be driven into the humerus 10 and through the channel 122 in a direction D2 to further secure the base 100 within the humerus 10. In the particular illustrated embodiment, the direction D2 is substantially parallel to (or even coaxial with) the intramedullary canal of the humerus 10.

FIG. 3C illustrates the secondary anchor 130 having been implanted into the humerus 10 after the base 100 has been implanted into the humerus. As noted above, secondary anchor 130 is implanted through a channel 122 in the anchor 120. In the illustrated embodiment, another channel 124 is provided in the collar 110. The channel 124 may be generally similar to the channel 122, and preferably (but not necessarily) is fully circumscribed by the collar 110. A central longitudinal axis that passes through channel 122 also preferably passes through the center of channel 124, so that channels 122 and 124 are coaxial. Thus, in order to implant secondary anchor 130 after base 100 is implanted, channel 124 in collar 110 may be used as a guide. Because the proximal surface of the collar 110 is exposed after implantation, the surgeon may be able to directly visualize the opening that leads to channel 124. The secondary anchor 130 may be passed through channel 124, with the secondary anchor 130 advancing distally and being guided into channel 122 by virtue of the alignment between channels 122, 124. In some embodiments, it may be possible to omit channel 124, although in such circumstances, guiding the secondary anchor 130 into the channel 122 may be more difficult and require guidance, such as via imaging.

Still referring to FIG. 3C, the combination of anchor 120 and secondary anchor 130 may help increase fixation and resist the tendency of the base 100 to pull out or otherwise loosen, particularly in the directions D3 and D4. Direction D3 is generally along the center longitudinal axis of the anchor 120, the opposite of D1, while D4 is generally along (or parallel to) the longitudinal axis of the humerus 10 (or the humeral intramedullary canal), the opposite of D2. If any forces in the direction of D3 are applied to the base 100, the secondary anchor 130 may particularly resist motion in that direction. And if any forces in the direction of D4 are applied to the base, the anchor 120 may particularly resist motion in that direction. Because the secondary anchor 130 is coupled to the base 100 (e.g. via collar 110 and/or anchor 120), the assembled construct of base 100 and secondary anchor 130 will be particularly effective in resisting any tendency to loosen or otherwise be pulled out of the bone after implantation. It should be understood then, that in order to remove the assembled construct (e.g. in preparation for a future joint replacement procedure), it may be necessary or at least desirable to first remove the secondary anchor 130, and only afterwards remove the base 100.

Although in the text above, the order of implantation is described as being the base 100 being implanted first, and the secondary anchor 130 being implanted second, in some embodiments the secondary anchor 130 may be implanted first, followed by the base 100. In that embodiment, it may be preferable to have channel 122 not fully circumscribed by anchor 120, or otherwise a different connecting mechanism between the anchor 120 and the secondary anchor 130 than is shown in connection with FIGS. 3A-C.

In some embodiments, no additional locking or fixation mechanism between secondary anchor 130 and base 100 is required beyond simply passing the secondary anchor 130 through channel 122 and/or 124. However, in other embodiments, alternative or additional locking mechanisms may be used. For example, tapered connections or press-fit relations between the secondary anchor 130 and the base 100 may provide fixation between the two components. Additionally or alternatively, adhesives such as biocompatible glue may help fix base 100 to secondary anchor 130. In other embodiments, threading may be provided to create a screw-type mechanism to fix the secondary anchor 130 to the base 100. For example, the secondary anchor 130 may include external threads and the channel 122 and/or 124 may include corresponding internal threads so that the secondary anchor 130 may be screwed into the base 100.

While secondary anchor 130 is illustrated as a relatively short member in FIGS. 3A-C, it should be understood that secondary anchor 130 may extend farther into humerus 10. For example, FIG. 3D illustrates secondary anchor 130 having a greater length than as shown in connection with FIGS. 3A-C. As shown in FIG. 3D, secondary anchor 130 may extend a significant length into the canal of the humerus 10, and including one or more apertures to receive fasteners 140 therethrough. For example, secondary anchor 130 may include one or more threaded holes that can received bones screws 140 therethrough, the bone screws being passed through the cortical shell of the humerus 10 and laterally (e.g. transverse the longitudinal axis of the intramedullary canal of the humerus 10) to provide additional fixation of the secondary anchor 130. If such a feature is utilized, it may be preferable to use a targeting device and/or image guidance to allow for the fasteners 140 to be passed into the humerus 10 in the desired position and trajectory in order to be received within the corresponding apertures of the elongated secondary anchor 130. It should be understood that fasteners 140 may provide even further fixation of the construct of the base 100 and the secondary anchor 130. In some embodiments, elongated secondary anchor 130 may be further fixed into the humerus 10 with the use of a cement mantle around the elongated secondary anchor 130. In fact, whether elongated or not, and whether fasteners 140 are provided, the secondary anchor 130 may be fixed to the humerus 10 with the use of bone cement or similar material.

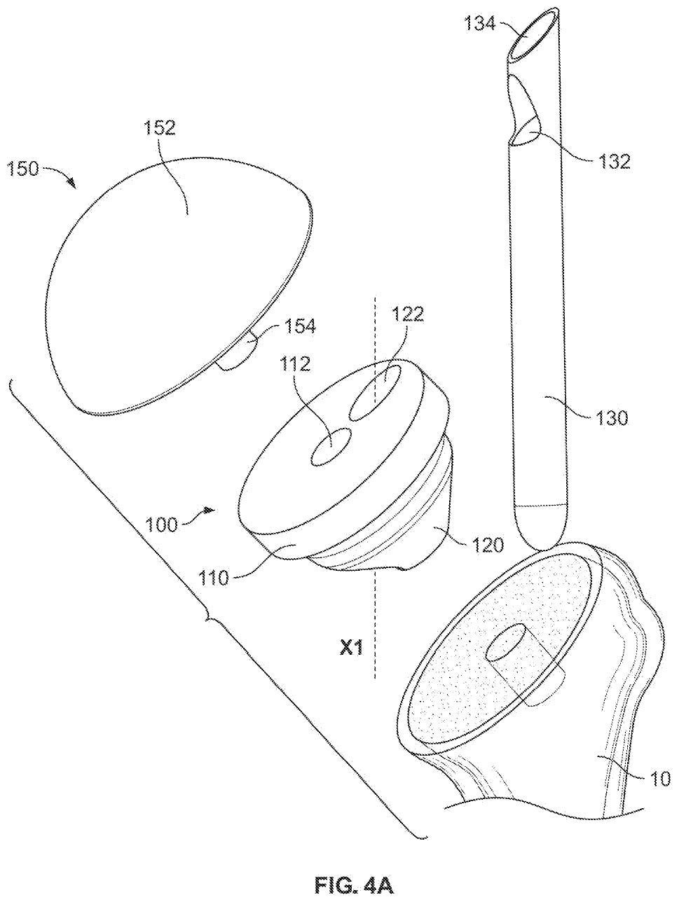

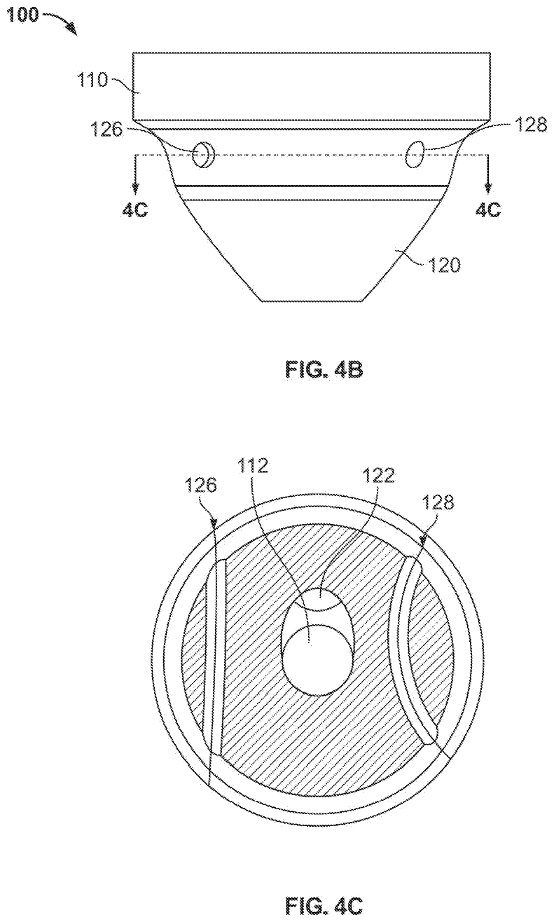

It should be understood that both the base 100 and the secondary anchor 130 illustrated in FIGS. 3A-D are shown as simplified components. The base 100 may take various shapes, including those shown and described in U.S. Patent Application Publication No. 2018/0271667 and U.S. patent application Ser. No. 17/308,107, the disclosures of which are hereby incorporated by reference herein, with modifications to provide for the desired interaction with the secondary anchor 130. For example, FIGS. 4A-C illustrate an embodiment of base 100 and secondary anchor 130 that provides additional specific details.

Referring to FIG. 4A, base 100 is illustrated, along with secondary anchor 130, the proximal end of humerus 10, and a prosthetic humeral head 150 to be coupled to base 100. Prosthetic humeral head 150 may include a convex surface 152 intended for articulation with a native or prosthetic glenoid. Prosthetic humeral head 150 may also include a connector 154 to be received within base 100. In the illustrated example, connector 154 includes a taper, such as a Morse taper, that is received within a corresponding tapered hole 112 in the collar 110 of base 100. FIG. 4A illustrates an opening in the collar 110 that leads to channel 122. In the illustrated embodiment, channel 122 may be continuous with channel 124 in anchor 120. In other words, channels 122 and 124 are not separate channels in FIG. 4A, but rather a single continuous channel that extends along an axis X1, the distal end of the channel 122 opening at anchor 120. As described above, the axis X1 of the channel 122 may be coaxial with, or parallel to, the intramedullary canal upon implantation of the base 100 into the humerus 10.

In the embodiment illustrated in FIG. 4A, the tapered hole 112 is in fluid communication with channel 122, as described in greater detail below. The secondary anchor 130 is illustrated as being generally cylindrical with a circular cross-section, and thus channel 122 may also be generally cylindrical with a circular cross-section. As noted above, however, secondary anchor 130 may have other shapes, including an oval or rectangular cross-section, with channel 122 having a corresponding cross-section. Depending on the cross-sectional shape of the secondary anchor 130 and the channel 122, the secondary anchor 130 may only fit into channel 122 in one, or a limited number of, rotational orientations. This alternative cross-sectional shape may also prevent rotation of the secondary anchor 130 relative to the base 100 after the components are assembled, which may be desirable. The secondary anchor 130 may include a cut-out or recessed section 132, as well as an angled proximal face 134. In one embodiment, the base 100 is first implanted into humerus 10 (with or without earlier preparation of the resected proximal humerus 10). With the base 100 implanted, both the tapered hole 112 and proximal opening of the channel 122 in the collar 110 are able to be directly visualized by the surgeon. The secondary anchor 130 may then be implanted into the humerus 10, for example into the intramedullary canal of the humerus 10, by passing the secondary anchor 130 through the channel 122. In some embodiments, the humeral canal may first be prepared to receive the secondary anchor 130, although such preparation is not required. The secondary anchor 130 is inserted until the proximal face 134 is substantially flush with the proximal face of collar 110. However, in other embodiments, the proximal face 134 of the secondary anchor 130 may extend a depth into the base 100. When the secondary anchor 130 is at the desired depth, the recessed portion 132 aligns with the tapered hole 112 so that, when the connector 154 of the prosthetic humeral head 150 is inserted into the tapered hole 112, the body of the secondary anchor 130 does not interfere with such insertion. Although tapered hole 112 is described as having a Morse taper to correspond to connector 154, it should be understood that in some embodiments, tapered hole 112 may be a non-tapered hole.

Although additional fixation of the secondary anchor 130 is not specifically required, it should be understood that fixation of the secondary anchor 130 may be achieved by bone cement, additional fasteners such as fasteners 140 illustrated in FIG. 3D, and/or via other locking mechanisms such as a threaded connection between the secondary anchor 130 and the base 100 as described above.

FIG. 4B is a side view of base 100, illustrating suture passages 126, 128. FIG. 4C is a cross-section of base 100 taken along the section line 4C-4C of FIG. 4B. FIGS. 4B-C illustrate optional suture passages 126, 128 passing through the base 100. As shown in FIG. 4B, each suture passage 126, 128 general opens to the outside of the base 100 at a position on anchor 120 just distal to the collar 110, although other positions relative to the collar 110 may be appropriate. As best shown in FIG. 4C, suture passage 126 is relatively straight extending mostly in a single direction transverse to the longitudinal axis of the anchor 120, whereas suture passage 128 has more curvature compared to suture passage 126. It should be understood that although two suture passages 126, 128 with specific trajectories are illustrated, suture passages may be omitted, or the base 100 may be provided with one, or more than two, suture passages. Further, any suture passages that are provided by have shapes and trajectories similar to, or different than, those shown in FIG. 4C.

Following implantation of base 100, sutures may be passed from outside the cortical shell of the humerus 10 through one of the suture passages 126, 128, until the suture passes through the opposite end of the suture passage 126, 128 and passes through the humerus 10 on the opposite side. A targeting device (similar to that used with fasteners 140) or other guiding (such as imaging) may be used to ensure that the sutures are positioned in the correct location and advanced with the desired trajectory to enter the suture passages 126, 128. The sutures that extend through the passages 126, 128 and outside the humerus 10 may be tied or otherwise fixed to provide enhanced fixation of the base 100 within the humerus 10, as well as to help prevent rotation of the base 100 relative to the humerus 10 after implantation. Although the term sutures is used, it should be understood that other materials, such as relatively rigid pins (e.g. strands of a metal or metal alloy such as nitinol) may be used with the suture passages 126, 128. The suture passages 126, 128 may also be used to help secure fractured portions of the humerus 10 to the base 100 and/or to the remaining portion(s) of the humerus 10. The curvature (or lack thereof) of the suture passages 126, 128 may be based on preference. For example, if a needle or other leading member attached to the sutures has a curvature, such a needle may more easily pass through suture channel 128 compared to suture channel 126.

It should also be noted that the cross-section of FIG. 4C illustrates the communication between the tapered hole 112 and the channel 122 described above.

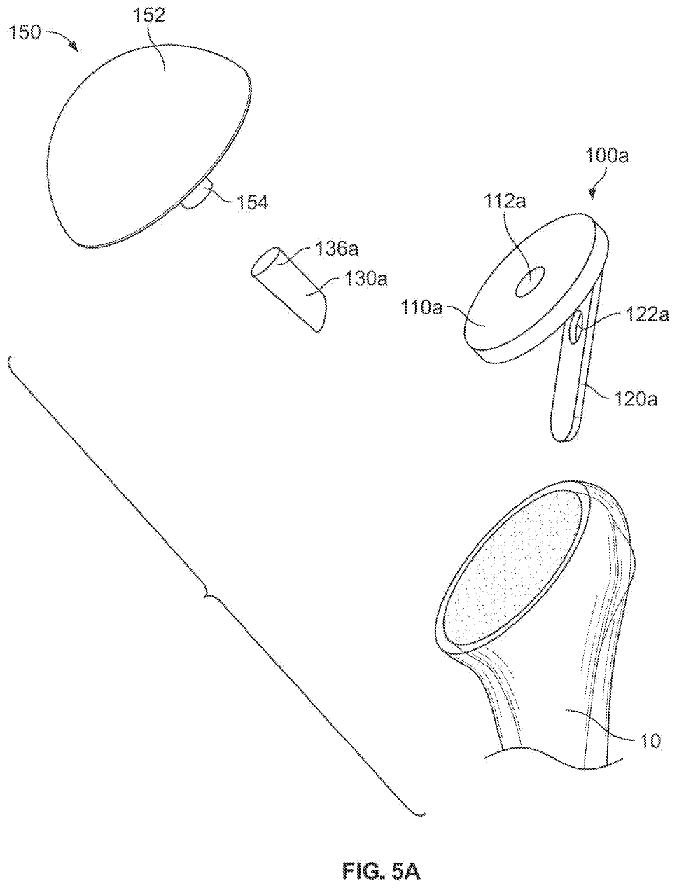

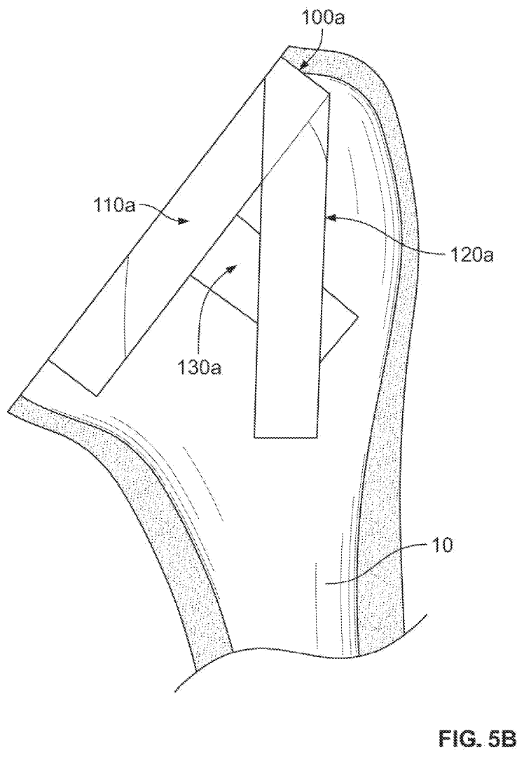

FIGS. 5A-B illustrate another embodiment of a humeral implant system, including prosthetic humeral head 150, base 100a, and secondary anchor 130a. As with other embodiments described herein, although the base 100a is illustrated as coupling with a prosthetic humeral head 150, the base 100a may also be used with a reverse shoulder implant system, in which case the base 100a may couple with a prosthetic cup (or tray) instead of a prosthetic humeral head 150. Prosthetic humeral head 150 may be identical to that described in connection with FIG. 4A and is thus not described again here.

Base 100a may include a collar 110a that is generally similar to collar 110 described above. For example, collar 110a may be a short cylinder with a circular cross-section, although other shapes may be suitable. The proximal face of collar 110a, when implanted into the humerus (for example as shown in FIG. 5B), is preferably substantially flush with the resected proximal surface of the humerus 10. The collar 110a may include an aperture 112a, for example near a center of the collar 110a, that extends from the proximal face through the distal face. Aperture 112a may be generally cylindrical, although other shapes may be suitable. Aperture 112a need not be tapered, but in some embodiments it may be tapered.

A main difference between base 100 and base 100a is the shape and position of the anchor 120 compared to the anchor 120a. As best shown in FIG. 5A, anchor 120a may have a proximal end that is attached to (or near) an outer circumference of the collar 110a. When the base 100a is implanted into the humerus 10, the proximal end of the anchor 120a may be positioned laterally (although in other embodiments other positioning including medial positioning may be suitable). The anchor 120a is a generally flat elongate member and may include a small thickness (in the medial-to-lateral direction) relative to its width (in the anterior-to-posterior direction). The anchor 120a may extend from its proximal point of attachment to collar 110a to a free distal end, such that the anchor 120a extends at an oblique angle relative to the proximal and distal faces of the collar 110a to form a general “V”-shape with the collar 110a. In some embodiments, the anchor 120a may include an aperture 122a, which may receive a component such as secondary anchor 130a, described in greater detail below.

In use, the collar 110a may be implanted into the humerus 10 first, with the distal or free end of the anchor 120a leading the implant, until the proximal face of the collar 110a is substantially flush with the proximal resection of the humerus 10. As best shown in FIG. 5B, the “V”-shape of the collar 110a and anchor 120a construct results in a volume of native bone being positioned between the two components. In fact, as the anchor 120a drives into the humerus 10, a volume of native humeral bone (e.g. cancellous bone) may compress between the anchor 120a and the collar 110a. The native volume of bone of the humerus 10 trapped between the anchor 120a and the collar 110a may provide significant resistance against the base 100a pulling out of the humerus 10, particularly in a direction orthogonal to the proximal face of the collar 110a. In order to position the anchor 120a within the humerus 10, a corresponding cavity may be created in the bone using a reamer, punch, or other suitable bone preparation tool.

After the base 110a is implanted, the secondary anchor 130a may be implanted. The secondary anchor 130a may be a generally cylindrical member, and the distal or leading end may be passed through aperture 112a of the collar 110a, until the distal or leading end of the secondary anchor 130a is received within aperture 122a of anchor 120a, as best shown in FIG. 5B. The proximal or trailing end of the secondary anchor 130a may include an aperture, such as tapered hole 136a, which may receive the connector 154 of the prosthetic humeral head 150. As noted above, the tapering may be a Morse taper to help secure the prosthetic humeral head 150 to the assembled base 100a and secondary anchor 130a. Preferably, the secondary anchor 130a is inserted in a direction orthogonal to the proximal face of the collar 110a. When implanted and assembled as shown in FIG. 5B, the anchor 120a may be particularly helpful in resisting pull-out of the base 100a in a direction orthogonal to the proximal face of the collar 110a, while the secondary anchor 130a may be particularly helpful in resisting pull-out of the base 100a in a direction parallel to the intramedullary canal of the humerus 10.

The anchor 120a may be formed monolithically (or integrally) with the collar 110a. However, in other embodiments, the anchor 120a and collar 110a may be formed separately, implanted separately, and only coupled during/after implantation. For example, the anchor 120a may be provided as a separate member implanted first, and the collar 110a may be implanted next, with mating features provided to couple the collar 110a to the anchor 120a. In other embodiments, the collar 110a and anchor 120a may be coupled to each other in any suitable fashion, such as via adhesives, during or after implantation.

The fixation of the base 100a may be increased by providing for an amount of flexion of the anchor 120a to compress native bone between the anchor 120a and the collar 110a. For example, if the aperture 122a is provided with a taper (such as a Morse taper), and the outer surface of the secondary anchor 130a is provided with a corresponding taper, the secondary anchor 130a may be driven farther through aperture 122a to cause flexing (e.g. “pulling”) of the anchor 120a toward the collar 110a as the secondary anchor 130a is driven farther through the aperture 122a. Additionally or alternatively, the anchor 120a may be flexed in one direction (or provided with bias otherwise) and implanted while flexed/biased, so that the anchor 120a tends to revert to a non-biased condition. The tendency to revert to the non-biased condition may cause native bone to compress farther. For example, the anchor 120a may be flexed laterally during implantation, and then attempt to “unflex” medially after implantation to cause compression of bone between the anchor 120a and the collar 110a, which may provide for additional fixation of the base 100a within the humerus 10.

The angle between the anchor 120a and the collar 110a may be provided as a single angle, or a group of bases 100a may be provided, each with a different angle between the proximal face of the collar 110a and the extension direction of the anchor 120a. Having a set of bases 100a with different angles may allow a user to choose the best angle for achieving the best fixation for a particular patient. In other embodiments, the angle between the collar 110a and the anchor 120a may be patient-specific, either based on a single individual patient's anatomy, or based on a set of patient data. The length of the anchor 120a may be similarly provided as a single value, different values within a kit of bases 100a, or specific to a single patient or specific based on analysis of a group of patients. For example, a particular patient's humerus 10 may be scanned (e.g. via CT or MRI imaging) to determine the geometry of the bone, as well as the quality of the bone. For example, the imaging may help to distinguish the density of the bone at different locations (e.g. based on the brightness of the pixels in the imaging). Based on the patient's imaging results, the length and/or angle of the anchor 120a may be customized so that the anchor 120a is located in the best bone quality for providing anchoring, and while ensuring that the anchor 120a is not at risk of penetrating through the cortical shell of the bone upon implantation. While individual patient imaging may be suitable for designing a patient-specific base 100a, a database may also be used. For example, the Stryker Orthopaedic Modeling and Analytics (“SOMA”) database may be leveraged to determine, across a patient population, optimal lengths and/or angles of the anchor 120a for achieving optimal fixation without penetrating the cortical shell of the humerus 10.

Although secondary anchor 130a is described as having a tapered fit with aperture 122a, other fits may be suitable. In one example, secondary anchor 130a may be have external threading and aperture 122a may have internal threading to allow for a screw-type connection between secondary anchor 130 and anchor 120a. In some embodiments, secondary anchor 130a may have a proximal flange or shoulder that limits the depth that he secondary anchor 130a may be inserted through aperture 112a. In such embodiments, additional screwing of secondary anchor 130a into aperture 122a may tend to pull the anchor 120a toward the collar 110a, compressing bone between the anchor 120a and the collar 110a.

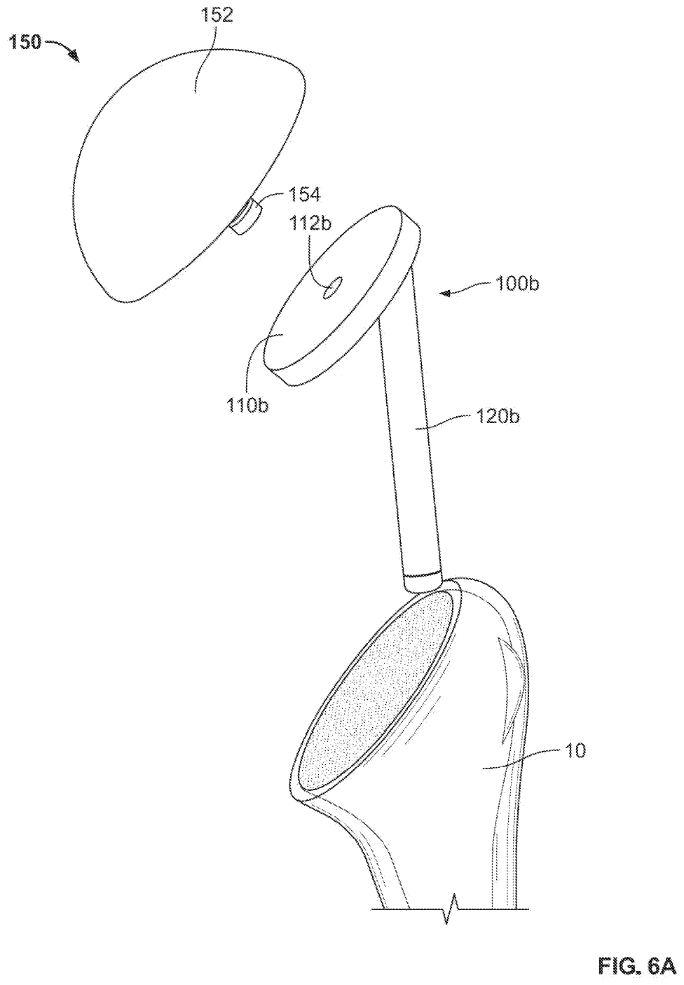

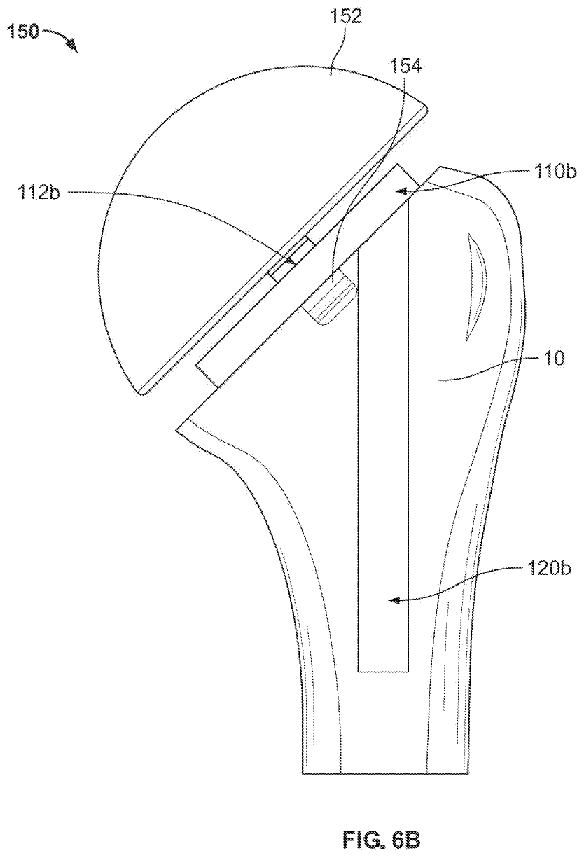

FIGS. 6A-B illustrate another embodiment of a humeral implant system, including prosthetic humeral head 150 and base 100b. As with other embodiments described herein, although the base 100b is illustrated as coupling with a prosthetic humeral head 150, the base 100b may also be used with a reverse shoulder implant system, in which case the base 100b may couple with a prosthetic cup (or tray) instead of a prosthetic humeral head. Prosthetic humeral head 150 may be similar or identical to that described in connection with FIG. 4A and is thus not described again here.

Base 100b may include a collar 110b that is generally similar to collars 110, 110a described above. For example, collar 110b may be a short cylinder with a circular cross-section, although other shapes may be suitable. The distal face of collar 110b, when implanted into the humerus (for example as shown in FIG. 6B), is preferably substantially flush with the resected proximal surface of the humerus 10. In other words, whereas collar 110a of base 100a is preferably flush with the resected proximal surface of the humerus 10, collar 110b of base 100b may effectively rest atop the proximal surface of the humerus 10. With this approach, minimal bone may be removed from the proximal humeral resection compared with other embodiments described here.

The collar 110b may include an aperture 112b, for example near a center of the collar 110b, that extends from the proximal face through the distal face. Aperture 112b is preferably, but need not be, tapered. The connector 154 of the prosthetic humeral head 150 may include a male taper (e.g. a Morse taper) that fits with a complementary taper in aperture 112b to help lock the prosthetic humeral head 150 to the base 100b.

Base 100b may include an anchor 120b. In the illustrated embodiment, anchor 120b includes a generally cylindrical main body and extends to a blunted terminal end. In use, the anchor 120b is implanted into, and generally coaxial with, the intramedullary canal of the humerus 10. In some embodiments, the anchor 120b may be configured to have a press-fit engagement with the intramedullary canal without additional fixation. However, in other embodiments, additional or alternative fixation modalities may be used. For example, the anchor 120b may be fixed within the intramedullary canal of the humerus 10 with cement or other suitable adhesive. Other options, such as the fasteners 140 described in connection with FIG. 3D, may additionally or alternatively used with anchor 120b. It should be understood that if fasteners 140 are utilized, corresponding apertures would be provided within anchor 120b.

Preferably, the anchor 120b and the collar 110b are formed as a single integral or monolithic construct. However, in other embodiments, the anchor 120b and the collar 110b may be formed as separate members and attached to each other prior to, or during, implantation.

In use, the anchor 120b is first implanted into the intramedullary canal of the humerus 10, either with or without additional fixation mechanisms such as cement. After positioning the anchor 120b, the collar 110b is positioned to rest on the plane of the proximal resection of the humerus 10. If anchor 120b and collar 110b are monolithic, these pieces are implanted simultaneously. If anchor 120b and collar 110b are separate pieces, they may either be coupled together first and then implanted together, or the anchor 120b may be implanted first, and then the collar 110b may be positioned and coupled to the anchor 120b.

In some embodiments, the collar 110b may be separately fixed to the humerus, for example using fasteners such as screws or cross-pins, adhesives such as cement, and/or a press-fit with the humeral bone. However, in other embodiments, additional fixation of the collar 110b is not required.

After the base 100b is in the desired position, prosthetic humeral head 150 (or a humeral cup or tray for a reverse shoulder arthroplasty procedure), may be coupled to the collar 110b by inserting connector 154 through aperture 112b. As shown in FIG. 6B, connector 154 may extend beyond the distal end of the collar 110b and extend directly into the bone of the humerus 10. In other embodiments, the connector 154 (or a connector of a humeral cup or tray component) may be elongated so that, upon insertion through aperture 112b, the connector 154 may engage with the anchor 120b. For example, the anchor 120b may be provided with an aperture or other mating feature, which may be similar to the aperture 122a of anchor 120a of base 100a, so that the connector 154 may directly lock into anchor 120b, providing even further fixation.

As shown in FIG. 6B, when base 100b is implanted, the collar 110b is positioned generally parallel to the proximal resection of the humerus 10, while the anchor 120b is positioned generally parallel to (and/or coaxial with) the axis of the intramedullary canal of the humerus 10. This positioning results in the anchor 120b having an oblique angle relative to the proximal and/or distal faces of the collar 110b. This angle, and the fact that the base 110b and anchor 120b are not able to move relative to each other, helps to minimize the risk of subsiding of the base 100b and/or micromotion of the base 100b after implantation.

As described for all of the embodiments above, although the bases of the humeral implant system are shown as engaging with a prosthetic humeral head, the bases could be used (with or without modification) to engage with components of a reverse shoulder arthroplasty system, such as a tray or a cup intended to interact with a glenoid implant, such as a glenosphere. But it should be further understood that the bases described herein are not limited to use in the humerus or in shoulder implants. For example, the bases described herein may be implanted into any suitable long bone for a joint replacement procedure, such as the femur for a hip replacement.

Further, although the structure of various bases have been described herein, it should be understood that the bases may be provided with additional features as desired. Any structure of the bases described herein, particularly those that will be in direct contact with bone, may be provide with features to enhance the fixation with the bone. For example, surfaces that will be in direct contact with bone may be provided with roughened surfaces, for example a porous metal surface, in order to enhance bone ingrowth into those surfaces to achieve better fixation over time. Similarly, structures such as flutes, serrations, or pegs may also be provided on surfaces that will engage bone to provide for increased fixation where desired and appropriate.

Some of the benefits of the implant systems described herein have already been described above, including the enhanced fixation provided by the bases. However, other benefits may also be achieved. For example, while good fixation is generally important for all prosthetic joint devices, reverse shoulder arthroplasty procedures may represent some of the worst case implants in terms of loading conditions. For example, a humeral component of a reverse shoulder arthroplasty system may generally experience more compression and higher sheer stresses compared to a humeral component of a total shoulder arthroplasty system. The bases described herein may provide high levels of resistance to both pull-out and torque-out of the humeral component, which may be especially desirable for a reverse shoulder arthroplasty procedure.

Still further, the humeral base component described herein may be well-suited for use in patients with very poor bone quality (e.g. lower than normal bone density), with little or no cancellous bone, and/or in revision cases in which a prior implant must be explanted and significant bone stock is missing after the explantation. The bases described herein may provide enhanced fixation and resulting better stability, even if the condition of the bone is poor. In these scenarios, the bases described herein may also increase the likelihood of achieving suitable fixation without the use of bone cement or other adhesives, which may be a desirable result.

For all of the bases described herein, any necessary bone preparation may be performed manually or with the help of either semi-autonomous or autonomous robotics. The use of a robotic system, such as the MAKO robot, may provide additional benefits in preparing the bone to receive the bases described herein, as the preparation of any desired bone cavity or recess may be performed with extreme precision to provide an exact match of implant geometry and bone shape. However, in some scenarios, little or no bone preparation may be needed once the initial resection (e.g. the proximal humeral resection in a shoulder arthroplasty) is performed.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims. For example, features described in connection with one embodiment may be combined, if suitable, with features described in connection with other embodiments.

1. A prosthetic implant system comprising:
a first articulation component;
a base having a proximal portion and a first anchor extending in a distal direction along a longitudinal first anchor axis, the proximal portion of the base configured to couple to the first articulation component; and
a second anchor formed separately from the base, the second anchor extending along a longitudinal second anchor axis;
wherein the base includes a channel extending from a first opening in the proximal portion of the base through a second opening in a distal portion of the first anchor, the channel sized and shaped to receive the second anchor therethrough;
wherein when the second anchor is received within the channel, the longitudinal first anchor axis is oblique to the longitudinal second anchor axis.

2. The prosthetic implant system of claim 1, wherein the proximal portion of the base includes an aperture, and the first articulation component includes a connector, the aperture being sized and shaped to receive the connector therein.

3. The prosthetic implant system of claim 2, wherein the aperture opens to, and is in fluid communication with, the channel.

4. The prosthetic implant system of claim 3, wherein the second anchor includes a recessed portion, and when the second anchor is received within the channel, the aperture opens to, and is in fluid communication with, the recessed portion of the second anchor.

5. The prosthetic implant system of claim 1, wherein the second anchor is circular in cross-section and the channel is circular in cross-section.

6. The prosthetic implant system of claim 1, wherein the second anchor is oblong in cross-section and the channel is oblong in cross-section, so that when the second anchor is received within the channel, the second anchor is prevented from rotating about the longitudinal second axis.

7. The prosthetic implant system of claim 1, wherein the second anchor has a regular polygon shape in cross-section, and the channel has a corresponding regular polygon shape in cross-section, so that when the second anchor is received within the channel, the second anchor is prevented from rotating about the longitudinal second axis.

8. The prosthetic implant system of claim 1, wherein the proximal portion of the base is substantially planar.

9. The prosthetic implant system of claim 8, wherein the second anchor includes a substantially planar proximal end oriented at an oblique angle relative to the longitudinal second axis, so that when the second anchor is received within the channel, the substantially planar proximal portion of the base is flush with the substantially planar proximal end of the second anchor.

10. The prosthetic implant system of claim 1, wherein the base includes a first suture passageway extending through the base in a first direction transverse the longitudinal first axis, the first suture passageway being circumscribed by the base and defining two openings at opposite ends of the first suture passageway.

11. The prosthetic implant system of claim 10, wherein the base includes a second suture passageway extending through the base in a second direction transverse the longitudinal second axis, the second suture passageway being circumscribed by the base and defining two openings at opposite ends of the first suture passageway.

12. The prosthetic implant system of claim 11, wherein the first suture passageway has a curvature that is different than a curvature of the second suture passageway.

13. The prosthetic implant system of claim 1, further comprising:
a fastener;
the second anchor including a first opening oriented generally transverse the longitudinal second axis, the first opening sized and shaped to receive the fastener therein.

14. The prosthetic implant system of claim 13, wherein in an assembled condition of the prosthetic implant system, the fastener is received within the first opening, and the fastener is positioned distal to the first anchor.

15. The prosthetic implant system of claim 1, wherein first opening in the proximal portion of the base extends along a longitudinal opening axis, the longitudinal opening axis being oblique to the longitudinal first anchor axis.

16. The prosthetic implant system of claim 15, wherein the distal portion of the first anchor is spaced from the proximal portion of the base so that a void area is formed therebetween, the channel extending across the void area.

17. The prosthetic implant system of claim 16, wherein when the second anchor is received within the channel, the longitudinal opening axis is coaxial with the longitudinal second anchor axis.

18. The prosthetic implant system of claim 17, wherein a proximal end of the second anchor including an opening therein, and the first articulation component includes a connector, opening in the proximal end of the second anchor being sized and shaped to receive the connector therein.

19. The prosthetic implant system of claim 17, wherein when the second anchor is received within the channel, a proximal end of the second anchor is positioned within the first opening in the proximal portion of the base, and a distal end of the second anchor is positioned within the second opening in the distal portion of the first anchor.

20. The prosthetic implant system of claim 1, wherein the first articulation component is a prosthetic humeral head, and the prosthetic implant system is a total shoulder arthroplasty system.
